(19) United States
(12) Patent Application Publication
(10) Pub. No.: US 2006/0161043 A1
(43) Pub. Date: Jul. 20, 2006

(54) Drive for an endoscope

(76) Inventors: Martin Neumann, Uttenreuth (DE); Karl Heinz Bayer, Kleinsendelbach (DE)

Correspondence Address: LUCAS & MERCANTI, LLP, 475 PARK AVENUE SOUTH, 15TH FLOOR, NEW YORK, NY, 10016, US

(21) Appl. No.: 11/330,465

(22) Filed: Jan. 12, 2006

(30) Foreign Application Priority Data
Jan. 18, 2005 (DE) DE102005002461.0

(51) Int. Cl. A61B 1/00

(52) U.S. Cl. 600/114; 600/102; 600/101

(57) ABSTRACT

The drive (1) for a flexible endoscope (2) has support elements (3, 4) which move relative to one another in a displacement direction (s) perpendicular to the axial direction (a) of the endoscope (2) and can be fixed in a desired position. On each of the support elements (3, 4), there are drive rollers (5, 6, 7, 8) that drive the endoscope (2). The drive rollers (5, 6, 7, 8), are driven by an electric drive (10). The electric drive (10) is connected to a pedal (11) that controls the speed of rotation and/or direction of rotation of the drive rollers (5, 6, 7, 8). To improve the operating comfort for the physician, the support elements (3, 4) can adjusted relative to one another by means of a screw/thread element (14). The screw/thread element (14) having a release position in which the support elements (3,4) can be distanced from one another without actuation of the screw-thread element (14).

The invention relates to a drive for a flexible endoscope which is of tubular configuration in at least some sections, for the purpose of moving said endoscope in its axial direction, the drive of the endoscope comprising at least two support elements which can be moved relative to one another in a displacement direction perpendicular to the axial direction of the endoscope and can be fixed in a desired position, where, on each of the at least two support elements, there is arranged in each case at least one drive roller whose axis is arranged substantially perpendicular to the axial direction of the endoscope and substantially perpendicular to the displacement direction, the at least one drive roller being designed to frictionally engage and drive the endoscope arranged between the drive rollers, and at least one of the drive rollers, preferably all the drive rollers, being driven by a controllable or variable electric drive, said electric drive being connected to a pedal whose actuation travel and/or actuation direction influences the speed of rotation and/or direction of rotation of the drive rollers.

To perform diagnostic and therapeutic exploratory procedures, flexible endoscopes are used which are well known in the prior art. They are introduced orally or rectally and are advanced to the site that is to be inspected or are pushed along the area that is to be inspected, for example into the area of the large intestine (colonoscopy), the duodenum, the stomach (gastroscopy) or the esophagus.

The directional control of the endoscope tip, which can be moved in two planes, is effected mechanically by means of two control wheels on the handle of the endoscope, in order to illuminate the lumen as centrally as possible and protect the wall of the intestine or in order to permit optimal adjustment around a target region. The advance movement and withdrawal movement of the endoscope are performed manually, one of the operator's hands having to release its hold on the control wheels at the endoscope handle in order to grip the endoscope shaft and push it forward. To allow the operator to concentrate on the important functions in the handle area of the endoscope, namely two control wheels and the instrument operation at the opening of the working channel, this function is often also undertaken by the personnel members who are assisting in the endoscopy procedure. An important point in illuminating the lumen is to protect the wall of the intestine as much as possible in order to avoid causing the patient pain and in order to avoid damage to the wall of the intestine or, in the worst case, a perforation of the intestine.

An endoscope drive of the type mentioned in the introduction is known from U.S. Pat. No. 5,779,623. With the drive described there, an endoscope can be advanced in the axial direction, the drive being controlled by pedals which are actuated by the operator.

A similar drive for an endoscope is known from US 2004/0097789 A1. U.S. Pat. No. 6,726,675 describes a drive with which a catheter can be advanced in its axial direction into the body of a patient. In order to avoid damage to the wall of the intestine during the endoscopy procedure, it is known, from DE 42 42 291 A1 and from DE 199 20 717 A1, to use an inverted tube whose outer area is stationary relative to the wall of the intestine, and whose inner area follows the movement of the endoscope. For optimal adaptation to the varying width of the intestinal lumen, it is also possible, with this system, to use liquid to alter the volume between the outer and inner walls of the inverted tube. For this inverted tube system, a drive unit is also disclosed generally in the form of electronically activatable drive wheels which can be arranged in a housing around the inverted-tube system and transmit a driving force to the inverted tube and to the endoscope.

DE 101 41 226 A1 also discloses a guide system for endoscopes which consists of a housing arranged concentrically around the endoscope shaft, with rotatably mounted drive sleeves permitting both a transverse movement and also a rotational movement of the endoscope shaft.

DE 101 41 225 A1 discloses an endoscope guidance system which comprises a mechanical holding and guiding arm and which is made up of a number of hinged connections, a parallelogram guide and drive units.

In difficult operating conditions in particular, it is very important to be able to advance the endoscope with fine precision and in so doing have control of the forces needed for the advance movement, in order to avoid unnecessary pain, damage to the intestinal wall, or even perforation of the intestine.

These risk factors and the individual assessment of the point from when the advance force could become dangerous are directly related to the practical experience and training of the particular operator.

The devices described in the prior art still do not afford an optimum solution in this respect. Rather, it is still generally necessary to advance the endoscope manually, at least in critical areas, in order to be able to rule out the chance of injuring the patient.

If the function of advancing and withdrawing the endoscope is taken over by the operator himself, he must at this point take one hand away from the control wheels on the endoscope handle and interrupt the process of directional control of the endoscope tip. In endoscopic intervention procedures in particular, that is to say when instruments, for example biopsy forceps or injection needles, have to be operated by one hand via the work channel in the handle area, it is very inconvenient if, for precision control and exact movement of the endoscope to the pathological region of the intestinal wall, one hand additionally has to be used to push the endoscope forward. If the forward and rearward movement of the endoscope is taken over by the assistant personnel, it is an important condition that the team carrying out the examination (physician and assistants) is well coordinated and, above all, that there is good communication between the persons involved.

However, a disadvantage of this is also that the assistant is taken up with insertion of the endoscope and is unavailable for other work, for example care/monitoring of the patient, preparation of instruments, etc.

In light of the above problems and disadvantages, the object of the invention is therefore to develop an endoscope drive of the type in question in such a way that the stated disadvantages can be avoided. The aim is therefore to make it possible to move the endoscope forward and rearward with precision and in a controlled manner, such that injury to the patient can be ruled out. However, the physician carrying out the examination is to be able to achieve optimal directional control of the endoscope tip while keeping his hands on the control wheels of the endoscope handle.

According to the invention, this object is achieved by the fact that the at least two support elements can be moved and/or adjusted relative to one another by means of a screw/thread element, said screw/thread element having a release position in which the at least two support elements can be distanced from one another without actuation of the screw/thread element.

With this configuration, it is possible, if so required, to switch very quickly to conventional manual advance of the endoscope, if this proves necessary on account of special circumstances. The endoscope is then uncoupled from its clamping by the drive rollers and is manipulated by hand.

With this configuration, it is therefore possible, in a particularly simple manner, to insert the endoscope into body cavities with very great precision, while still ensuring that the physician can keep both hands on the control handle of the endoscope.

In a first development, the drive is designed for moving the endoscope in both axial directions.

The pedal and/or the controllable or variable electric drive can be designed for stepless movement of the endoscope.

To rule out the possibility of the patient being injured by the endoscope because the advancing force is too great, force sensors can be provided with which it is possible to measure the axial force applied to the endoscope by the at least one drive roller. The force sensors can comprise at least one strain gauge. Moreover, display means can be provided for displaying the axial force applied to the endoscope by the at least one drive roller. The display means can have optical elements, in particular light-emitting diodes, which have different colors in different ranges. This permits simple and clear monitoring of how great the advance force is. The display means can also or alternately comprise acoustic elements in order to provide an acoustic indication of an inadmissibly large increase in the advance force. Another alternative is that the display means comprise elements emitting vibrations, by means of which increasing advance forces are made noticeable. The force sensors can be connected to a switch element which is designed to switch off the electric drive if a predetermined maximum value of the axial force applied to the endoscope by the at least one drive roller is exceeded.

The screw/thread element can be designed for mirror-image actuation of the at least two support elements with respect to a plane of symmetry.

The drive rollers can comprise a covering made from a material with a high coefficient of friction, in particular from rubber. The rollers preferably have a conical outer periphery. Two interacting drive rollers can in each case be arranged in such a way that their conical outer peripheries are oriented in opposite directions.

The pedal can be equipped with two foot panels, one being provided for advancing the endoscope in one axial direction, and one being provided for withdrawing the endoscope in the other axial direction.

The support elements and drive rollers are preferably freely accessible. At least the drive rollers can be designed such that they can be coupled to the rest of the drive device and can be uncoupled from it. The drive rollers can in particular be arranged on a module. With this configuration, the rollers can be easily removed from the drive, for example via a quick-coupling means, and cleaned. A collecting dish can be arranged under the module and is sealed off from the support elements. In this way too, simple cleaning and hygienic handling of the entire drive is made possible. Wash means are also expediently provided for cleaning the endoscope.

Finally, the drive can comprise position sensors for measuring the advance and withdrawal of the endoscope.

The proposal according to the invention is therefore that, for conventional flexible endoscopes of different diameters, the advance movement and withdrawal movement in diagnostic and/or therapeutic interventions in the intestine/esophagus/stomach/duodenum are effected by an electronically controlled drive with foot control, with which variable speeds both in the insertion direction and in the withdrawal direction can be steplessly regulated, the advance force being able to be measured and displayed, and, if a predetermined limit on the force is exceeded, the forward movement of the endoscope is automatically switched off.

For this purpose, provision is made for the manual advance and withdrawal of the conventional endoscopes to be replaced by a foot-controlled electric drive which can be actuated by the operator himself so that he is able to concentrate on controlling and operating the instruments in the handle area. Moreover, the proposal according to the invention allows the assistant to concentrate on other important duties.

By means of the switching-off of the endoscope drive when a predetermined advance force is exceeded or when corresponding signals are emitted in the event of an increasing advance force, individual empirical values in respect of the admissible advance force during endoscopy can be objectified for younger physicians too and can be used as a guideline. The values determined can also be used to define optimal settings in respect of the pain burden for the patient.

Conventional, flexible endoscopes with different diameters can be used, without any need for special preparation or finishing of the sides of the endoscope. The drive rollers touch the endoscope with a force-fit engagement and without damaging its covering.

An illustrative embodiment of the invention is shown in the drawing, in which:

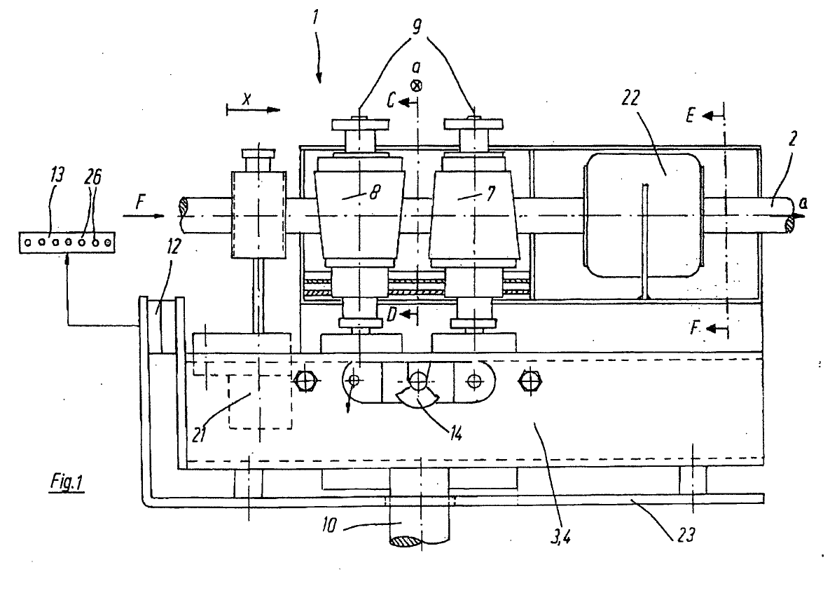

FIG. 1 shows the side view of the drive for an endoscope, for moving the latter in the axial direction,

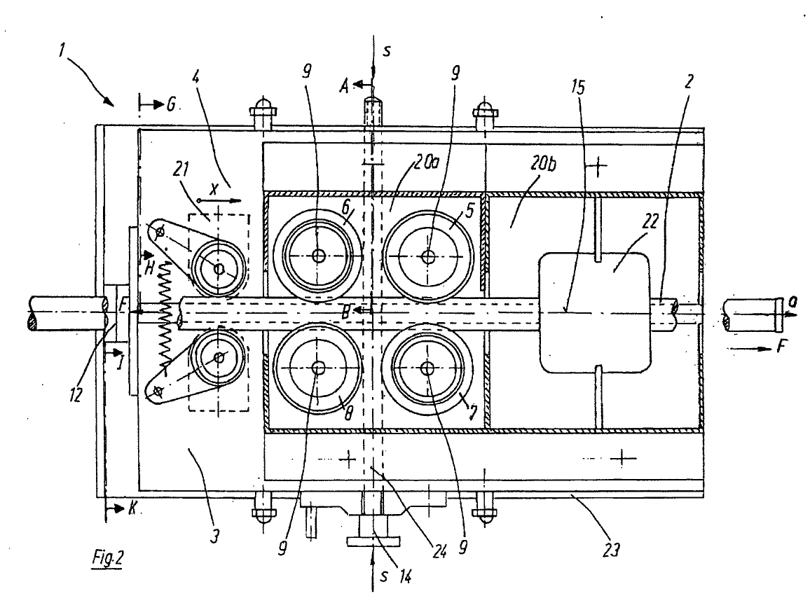

FIG. 2 shows the plan view corresponding to FIG. 1,

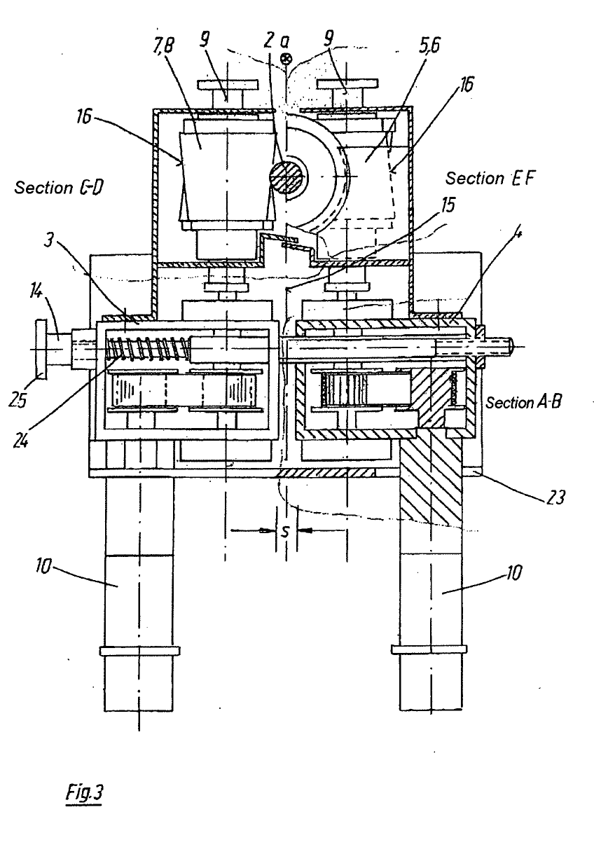

FIG. 3 shows the front view corresponding to FIG. 1 in cross section (cross sections C-D and E-F according to FIG. 1 and A-B according to FIG. 2),

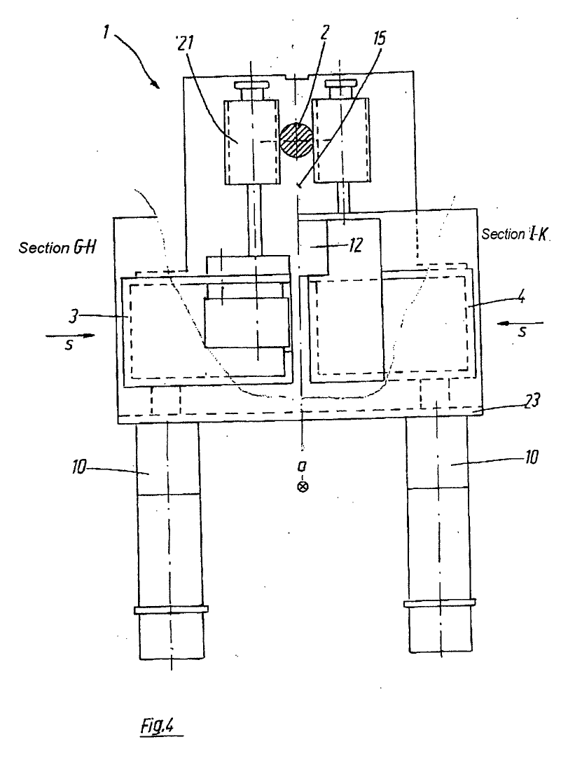

FIG. 4 shows the front view corresponding to FIG. 1 in cross section (cross sections G-H and I-K according to FIG. 2) and

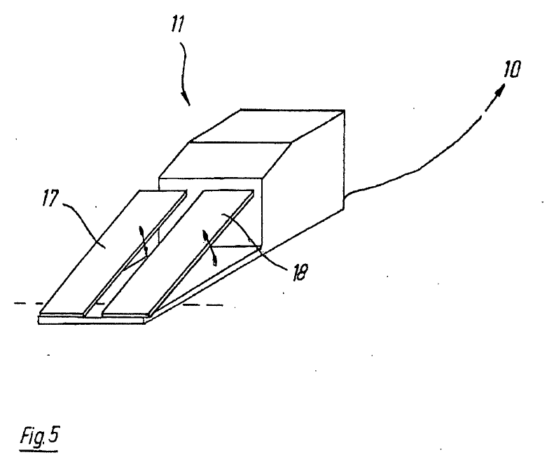

FIG. 5 shows the pedal of the drive in a perspective view.

The figures show a drive 1 for an endoscope 2 which can be moved in both axial directions along its axial direction a. The drive has two support elements 3 and 4 which are movable relative to the base frame 23 of the drive 1 and in mirror image with respect to a plane of symmetry 15 of the drive 1. The mirror-image movement of both support elements 3, 4 is effected by a screw/thread system 14 which comprises a threaded spindle 24 with restoring spring (which presses the two support elements 3, 4 apart) and with two thread sections, both thread sections being oriented in opposite directions, i.e. one thread section is designed as a right-hand thread and the other as a left-hand thread. The thread sections cooperate with threaded nuts, which are connected to the support elements 3, 4.

This has the effect that, when the threaded spindle 24 is turned by way of a handwheel 25, the two support elements 3, 4 are moved toward or away from one another. This displacement movement takes place in a direction s perpendicular to the axial direction a which is defined by the endoscope 2 when the latter is in the drive 1.

Arranged on the support elements 3, 4 there are a total of four drive rollers 5, 6, 7 and 8, the axis 9 of the rollers 5, 6, 7, 8 being perpendicular to the axial direction a and perpendicular to the displacement direction s.

As will be evident from a comparison of FIGS. 1, 2 and 3, two rollers 5 and 6 are arranged on the support element 4 and two rollers 7 and 8 are arranged on the support element 3. All the rollers have an outer periphery 16 with a conical profile. The endoscope 2 is clamped by frictional engagement by two rollers on each of the support elements 3, 4, such that, when the rollers 5, 6, 7, 8 turn, the endoscope 2 is displaced in axial direction a.

Two rollers clamping the endoscope 2- in the first instance the rollers 5 and 7 and in the second instance the rollers 6 and 8- are arranged in opposite directions in terms of their peripheral cone, as can be seen from FIG. 3. On the other hand, the adjacently arranged rollers 5, 6 and 7, 8 are also arranged in opposite directions in terms of their peripheral cone. The endoscope 2 is in this way kept centered in the position shown here.

The rollers 5, 6, 7, 8 are connected via electric drives 10, i.e. the rollers 5, 6, 7, 8 turn when the electric drives 10 are actuated. The electric drives 10 are controlled by a control system (not shown) which in turn receives its control signals from a pedal 11, see FIG. 5, operated by the physician.

In the illustrative embodiment, the pedal 11 has two foot panels 17 and 18, namely one for the advance of the endoscope 2 and one for the latter's withdrawal. The foot panels can in this case deliver to the control system a value proportional to the actuation angle of the foot panel 17, 18 (see arrows indicated in FIG. 5) such that the electric drives 10 are driven to a greater or lesser degree depending on the actuation angle of the foot panels 17, 18.

The pedal 11 with the two foot panels 17, 18 is therefore able to be finely controlled like a gas pedal; the right foot panel 18 can, for example, be used for driving the endoscope 2 forward, and the left foot panel 17 can correspondingly be used for its withdrawal.

Provision can also be made for the pedal 11 to have only a single foot panel which, for example, switches from advance to withdrawal by means of a pivoting movement about the vertical axis.

A very advantageous feature of the proposed drive for the endoscope is the adjustable or predeterminable limit on the axial advance force F applied to the endoscope 2 by the drive rollers 5, 6, 7, 8. In particular, it is possible to predetermine a maximum force Fmax which must not be exceeded. For this purpose, the drive 1 has force sensors 12 which comprise a bridge circuit of strain gauges which is known per se. As can be seen by way of example in FIG. 1, it is thus possible to measure the force F with which the endoscope 2 is advanced in axial direction a. This figure also indicates schematically that display means 13 are present for showing the current advance force F.

An electronic measurement of the advance force is also possible in principle, by measuring changes in resistance of the electric drives 10.

The force can in principle also be measured, not by measuring the pressure on the housing, but by arranging pressure sensors in the area of the endoscope tip which transmit a signal to the display means 13.

The display means 13 are conceived in particular as comprising a number of light-emitting diodes 26, more of which are switched on the greater the force. The first light-emitting diodes are green, the middle ones yellow, and the last ones red, so that, from the color of the light-emitting diodes, it is immediately possible to ascertain the degree of the advance force F. For example, provision can be made that resident physicians or interns are allowed to work only in the “green” range, that senior physicians are able to use the drive up to the “yellow” range, and that only chief physicians can venture into the red range, since, in the latter case, it must be assumed that special circumstances exist within the body cavity.

The insertion path x of the endoscope 2 (advance and withdrawal) into the body cavity can be determined by position sensors 21 (see in particular FIGS. 2 and 4) which can consist of at least one spring-tensioned roll bearing on the outer periphery of the endoscope 2.

Keeping the drive 1 clean is made easier by a hygiene module 19 (indicated schematically in FIG. 2), by which means it is possible to accommodate and sterilize all four drive rollers 5, 6, 7, 8 together. For this purpose, quick-coupling means (not shown in detail) can be provided between the drive rollers and the electric drives 10.

Arranged under the drive rollers 5, 6, 7, 8 there is a two-part collecting dish 20 (parts 20a and 20b in FIG. 2), which facilitates hygienic handling of the drive.

The collecting dish 20 serving for hygiene purposes has elastic feed-through seals which have to follow the travel in displacement direction s transverse to the axial direction a of the endoscope 2 when the latter is clamped, and the travel in axial direction a for force measurement.

Wash means 22, which are indicated only schematically in the figures, also serve for hygiene purposes. These may involve spray nozzles which spray the circumference of the endoscope with a cleaning liquid. The wash means can also comprises scraper rings for cleaning the withdrawn endoscope 2 which may possibly be soiled with fecal matter or mucosal residues.

An important functional feature is that the endoscope 2 is accessible from above, that is to say does not have a housing closing off the endoscope 2. Rapid and direct access to the endoscope is thus made possible.

With the aid of a quick-release means (quick coupling) for the screw/thread element 14 (not shown in detail), it is possible, in one maneuver, to release the endoscope 2 from its clamping by the drive rollers 5, 6, 7, 8 such that the endoscope 2 can, if necessary, be manipulated in the customary manual way.

List of reference labels drive endoscope support element support element drive roller drive roller drive roller drive roller axis electric drive pedal force sensors display means screw/thread element plane of symmetry outer periphery foot panel foot panel hygiene module collecting tray position sensors wash means base frame threaded spindle with restoring spring handwheel light-emitting diode axial direction s displacement direction axial force maximum axial force advance and withdrawal

1. A drive (1) for a flexible endoscope (2) which is of tubular configuration in at least some sections, for the purpose of moving said endoscope (2) in its axial direction (a), the drive (1) comprising at least two support elements (3, 4) which can be moved relative to one another in a displacement direction (s) perpendicular to the axial direction (a) of the endoscope (2) and can be fixed in a desired position, where, on each of the at least two support elements (3, 4), there is arranged in each case at least one drive roller (5, 6, 7, 8) whose axis (9) is arranged perpendicular to the axial direction (a) of the endoscope (2) and perpendicular to the displacement direction (s), the at least one drive roller (5, 6, 7, 8) being designed to frictionally engage and drive the endoscope (2) arranged between the drive rollers (5, 6, 7, 8), and at least one of the drive rollers (5, 6, 7, 8), preferably all the drive rollers, being driven by a controllable or variable electric drive (10), said electric drive (10) being connected to a pedal (11) whose actuation travel and/or actuation direction influences the speed of rotation and/or direction of rotation of the drive rollers (5, 6, 7, 8), wherein the at least two support elements (3, 4) can be moved and/or adjusted relative to one another by means of a screw/thread element (14), said screw/thread element (14) having a release position in which the at least two support elements (3, 4) can be distanced from one another without actuation of the screw/thread element (14).

2. The drive as claimed in claim 1, wherein the drive is designed for moving the endoscope (2) in both axial directions.

3. The drive as claimed in claim 1, wherein the pedal (11) and/or the controllable or variable electric drive (10) are designed for stepless movement of the endoscope (2).

4. The drive as claimed in claim 1, having force sensors (12) with which it is possible to measure the axial force (F) applied to the endoscope (2) by the at least one drive roller (5, 6, 7, 8).

5. The drive as claimed in claim 4, wherein the force sensors (12) comprise at least one strain gauge.

6. The drive as claimed in claim 4, having display means (13) for displaying the axial force applied to the endoscope (2) by the at least one drive roller (5, 6, 7, 8).

7. The drive as claimed in claim 6, wherein the display means (13) have optical elements, in particular light-emitting diodes, which have different colors in different ranges.

8. The drive as claimed in claim 6, wherein the display means (13) comprise acoustic elements.

9. The drive as claimed in claim 6, wherein the display means (13) comprise elements emitting vibrations.

10. The drive as claimed in claim 4, wherein the force sensors (12) are connected to a switch element which is designed to switch off the electric drive (10) if a predetermined value (Fmax) of the axial force (F) applied to the endoscope (2) by the at least one drive roller (5, 6, 7, 8) is exceeded.

11. The drive as claimed in claim 1, wherein the screw/thread element (14) is designed for mirror-image actuation of the at least two support elements (3, 4) with respect to a plane of symmetry (15).

12. The drive as claimed in claim 1, wherein the drive rollers (5, 6, 7, 8) comprise a covering made from a material with a high coefficient of friction.

13. The drive as claimed in claim 12, wherein the material is rubber.

14. The drive as claimed in claim 1, wherein the drive rollers (5, 6, 7, 8) have a conical outer periphery (16).

15. The drive as claimed in claim 14, wherein two interacting drive rollers (5, 7; 6, 8) are in each case arranged in such a way that their conical outer peripheries (16) are oriented in opposite directions.

16. The drive as claimed in claim 1, wherein the pedal (11) has two foot panels (17, 18), one being provided for advancing the endoscope (2) in one axial direction, and one being provided for withdrawing the endoscope (2) in the other axial direction.

17. The drive as claimed in claim 1, wherein the support elements (^{3, 4}) and drive rollers (5, 6, 7, 8) are freely accessible.

18. The drive as claimed in claim 1, wherein at least the drive rollers (5, 6, 7, 8) are designed such that they can be coupled to the rest of the drive device and can be uncoupled from it.

19. The drive as claimed in claim 18, wherein the drive rollers (5, 6, 7, 8) are arranged on a hygiene module (19).

20. The drive as claimed in claim 19, wherein a collecting dish (20) is arranged under the hygiene module (19) and is sealed off from the support elements (3, 4).

21. The drive as claimed in claim 1, wherein the drive comprises position sensors (21) for measuring the advance and withdrawal (x) of the endoscope (2).

22. The drive as claimed in claim 1, comprising wash means (22) for cleaning the endoscope (2).
